(19) United States
(12) Patent Application Publication
(10) Pub. No.: US 2017/0313605 A1
(43) Pub. Date: Nov. 2, 2017

(54) MODIFIED PROTEIN MATERIALS, METHODS AND USES THEREOF

(71) Applicant: THE GOVERNORS OF THE UNIVERSITY OF ALBERTA, Edmonton (CA)

(72) Inventors: David Curtis BRESSLER, Edmonton (CA); Paolo Giuseppe MUSSONE, Edmonton (CA); Lauren Gina MERCIER, Edmonton (CA)

(21) Appl. No.: 15/520,537

(22) PCT Filed: Oct. 21, 2015
(86) PCT No.: PCT/CA2015/051063

§ 371 Date: Apr. 20, 2017

Related U.S. Application Data
(60) Provisional application No. 62/066,676, filed on Oct. 21, 2014.

(51) Int. Cl. C02F 1/52; B01D 21/01; C07K 1/12; C07K 14/76; C07K 14/415; C02F 103/10

(52) U.S. Cl. CPC C02F 1/5263; C07K 14/76; C07K 14/415; B01D 21/01; C07K 1/12; C02F 2103/10

(57) ABSTRACT

Methods of modifying renewable protein sources and uses thereof are provided. In some embodiments, renewable protein sources can be modified to become a flocculant and/or coagulant through the use of a hydrolysis process. Further modifications can be performed in order to enhance the flocculant/coagulant ability of the modified protein material. Such modified protein material can be used to coagulate and/or flocculate waste water colloidal suspensions, either alone or in combination with a coagulant, by mixing the modified protein material with waste water colloidal suspensions to create a mixture and allowing mixture to settle. In some embodiments, the waste water colloidal suspension can be mature fine tailings (MFT).

### CROSS-REFERENCE TO RELATED APPLICATIONS

This application claims priority of U.S. Provisional Patent Application Ser. No. 62/066,676, entitled “Modified Protein Materials, Methods and Uses Thereof”, filed on Oct. 21, 2014, and hereby incorporated by reference herein in its entirety.

### TECHNICAL FIELD

The present application relates to protein materials, and more particularly, compositions and methods to modify and use modified protein materials.

### BACKGROUND

The rendering industry processes inedible tissues, offal, blood and bones from slaughtered farm animals to recover hides, purified fats and proteins. The outbreaks of bovine spongiform encephalopathy (BSE) in North America and Europe had a profound impact on the use of the protein fraction which previously was marketed as a feed ingredient for cattle, poultry, pets and aquaculture. The form of infectious agent thought to cause BSE and several transmissible spongiform encephalopathies (TSE) is a misfolded protein known as prion. Specified risk materials (SRM or SRMs), which include the skull, brain, trigeminal ganglia, eyes, spinal cord, and dorsal root ganglia from cattle over 30 months of age and the distal ileum and tonsils from cattle of all ages, are believed to be the highest risk material to contain prions in undiagnosed animals.

In Canada, SRM is rendered to recover lipids while the remaining fractions are landfilled. Currently, over three hundred thousand tonnes of such rendered SRM are disposed of to the landfill annually, posing economic challenges to the rendering industry with repercussions to the whole livestock industry. These challenges include costs attributed to segregation of SRM from non-SRM animal tissues, segregation of processing lines to handle SRM and non-SRM tissues, and costs associated with SRM storage, transporting and disposal fees. Disposal tipping fees range from $75 to $200 (depending on jurisdiction) per tonne and transportation costs are $250 per tonne on average. These circumstances constitute a strong drive to develop and market non-food/feed industrial applications from this waste stream.

The societal demand for chemical building blocks and products recovered or produced from low-value or waste streams, non-petroleum based renewable and sustainable resources is rapidly and consistently increasing. Furthermore, the need to reduce waste driven by expensive and restrictive waste disposal legislation and the escalating cost of raw materials constitute the necessity of recovering value from waste.

Despite progress, the full industrial implementation of novel applications of animal-derived protein, including SRM, remains hampered by the limited economic value of the derived products and by incomplete understanding of fundamental protein hydrolyzate molecular structure and properties.

In the Fort McMurray region of Northern Alberta, Canada, mine tailings resulting from the extraction of bitumen using the Clark Hot Water Extraction process are managed using settling basins. When the tailings stream reaches the pond, the heaviest fraction, which is primarily composed of sand, settles quickly while the aqueous fraction rises to the top. A middle layer, conventionally defined Mature Fine Tailings (MET), comprising a mixture of fine solid particles (clay and other minerals) and suspended hydrocarbons, forms a stable dispersion that is known to persist for decades. As of 2009, the Government of Alberta estimated that over 800 million m³ of MFT are deployed in tailing ponds affecting a total surface area exceeding 130 Km². Over thirty oil sands tailings treatment technologies have been developed since the inception of commercial bitumen extraction on a commercial scale. Oil sands operators are currently facing severe challenges in meeting environmental requirements both from a technical and economic standpoint.

Several technologies have been investigated and some are being tested at the pilot scale by industrial operators and academic research institutions. These include: the use of an integrated steam generation system, evaporators, freeze and thaw systems, in-situ densification via coke capping, CO₂ addition, centrifuge-based methods, thickening methods, and combined flocculation and filtration methods. In particular, the consolidated tailing (CT) technology, which uses gypsum to promote the coagulation of solids into trafficable cakes, has attracted much interest over the past decade. However, high costs associated with tailing handling (dredging, cycloning) and with difficulties in formulating an optimal surfactant mixture have hampered the widespread commercialization of this method. Another advanced, and increasingly more cost-effective approach, involves the integrated use of a surfactant system to segregate the solid fraction of the tailing from the aqueous phase followed by a spin dry cycle. The present main limitations of this approach are inter-related.

Flocculation is the aggregation of suspended particles to form discrete flocs. The coagulation and flocculation of suspended particles by chemical flocculants in liquid is of importance in many fields, such as water purification, waste water treatment, mineral processing, and tailings treatment in the oil sands industries. The most common industrial coagulants are inorganic agents such as ferric salts and aluminum sulfate, which produce a large amount of secondary sludge, resulting in toxicity and disposal issues. The most widely used flocculants are polyaluminum chloride (H. Seki, H.

Maruyama, Y. Shoji. Flocculation of diatomite by a soy protein-based bioflocculant. Biochemical Engineering Journal 51 (2010) 14-18, incorporated herein by reference) and high molecular weight polymers such as polyacrylamide. High-performance polymer flocculants (or their formulations) such as polyacrylamide are known to have overcome some of the difficulties (high dosage required, high costs) related to the initial use of calcium oxide or mono/bicarbonate flocculant systems.

However, such classes of anionic polymers, which have been developed to encompass various degrees of chain length and charge distribution, are known to have a negative impact on aquatic life. There are serious concerns about the lack of biodegradability and risk of environmental contamination associated with their use. For instance, in Germany sludges treated with polyacrylamide, whose monomers are known neurotoxins, will be excluded from disposal on areas under cultivation by the end of 2013 (D. C. Krentz, C. Lohmanna, S. Schwarzb, S. Bratskayac, T. Liebertd, J. Laubed, T. Heinzed, W. M. Kulickea, Properties and flocculation efficiency of highly cationized starch derivatives, Starch 58 (2006) 161-169, herein incorporated by reference). Moreover, the solid fraction is characterized by flocs that can not be consistently large and stable to be easily (and inexpensively) processed in a conventional filtration system.

As a result there has been a move in the area of waste water treatment to use bio-flocculants, which are both biodegradable and produce no secondary pollution. However, there remains a need to provide renewable, biodegradable, and environmentally benign methods and systems for the treatment of MFT that can overcome the limitations of the prior art.

### SUMMARY

Methods of modifying specified risk materials (SRM) and uses thereof are provided. In some embodiments, SRM can be modified to become a flocculant or coagulant, by providing hydrolyzed SRM and/or reacting carboxylic groups present in the hydrolyzed SRM with a low molecular weight alcohol to control the distribution of electrical charges on the hydrolyzed SRM. Such modified SRM can be used to coagulate and flocculate waste water colloidal suspensions, by mixing the modified SRM with waste water colloidal suspensions to create a mixture and allowing mixture to settle. In some embodiments, the waste water colloidal suspension can be mature fine tailings (MFT).

In some embodiments, the flocculant systems described herein can be renewable and environmental benign. In addition, the feedstock used to prepare the flocculant systems, can be SRM from the cattle rendering industry, which is a waste stream available at no/low cost as rendering operators are generally disposing of this material by landfilling it incurring in substantial costs.

Broadly stated, in some embodiments, a method is provided to coagulate and flocculate waste water colloidal suspensions, the method comprising: providing a flocculant comprising a modified protein material composition; mixing the flocculant with waste water colloidal suspensions to create a mixture; and allowing mixture to settle; wherein the waste water colloidal suspensions are coagulated and flocculated.

Broadly stated, in some embodiments, a use of modified protein material is provided for the coagulation and flocculation of mature fine tailings.

Broadly stated, in some embodiments, a method of modifying protein material is provided to become a flocculant or coagulant, the method comprising: providing hydrolyzed protein material; and reacting carboxylic groups present in the hydrolyzed protein material with an alcohol to control the distribution of electrical charges on the hydrolyzed protein material; wherein the hydrolyzed protein material is modified to become a flocculant or coagulant.

Broadly stated, in some embodiments, a modified protein material composition is provided, the composition comprising: hydrolyzed protein material comprising carboxylic groups and a controlled distribution of electrical charges; wherein the carboxylic groups were reacted with an alcohol to control the distribution of electrical charges.

Broadly stated, in some embodiments, a modified protein material is provided, comprising: hydrolyzed protein material selected from the group consisting of specified risk material (SRM), canola protein, albumin, gelatin, blood meal protein, and meat and bone meal protein, further comprising a controlled distribution of electrical charges; wherein the hydrolyzed protein material is water soluble; and wherein the hydrolyzed protein material acts as a flocculant of waste water colloidal suspensions. This modified protein material can further undergo any or all of the following treatments/processing: esterification, cationization and/or polymerization.

### BRIEF DESCRIPTION OF THE DRAWINGS

FIG. 1 depicts a flowchart outlining an embodiment of a method to modify renewable protein material;

FIGS. 2A-D depict the results of an embodiment of a method of flocculation activity of anionic polyacrylamide (PAM), gelatin, and SRM protein at A) 500 ppm, B) 400 ppm, 0) 250 ppm and D) 100 ppm concentrations;

FIG. 3 depicts the results of an embodiment of water release as a function of time with 400 ppm addition of SRM, Gelatine or PAM;

FIG. 4 depicts the results of an embodiment of a 24 hr SRM flocculation of MFT and control;

FIGS. 5A-C depict the particle size distribution pre-flocculation of A) original oil sand tailing and post-flocculation using an embodiment, specifically 400 ppm hydrolyzed SRM, of B) the sedimented residue, and C) the water released;

FIG. 6 depicts an embodiment of Surface Plot Turbidity (NTU) versus pH and the concentration of Calcium Chloride at a constant peptide value;

FIG. 7 depicts an embodiment of Surface Plot Turbidity (NTU) versus pH and the concentration of peptides recovered from SRM at a constant CaCl₂ value;

FIG. 8 depicts an embodiment of Surface Plot Turbidity (NTU) versus the concentration of Calcium Chloride and the concentration of peptides recovered from SRM at a constant pH value; and

FIG. 9 depicts an embodiment of weight loss of epoxy cured thermally hydrolyzed SRM based plastics as a result of natural soil and autoclaved soil burial for A) one month and B) three months.

### DETAILED DESCRIPTION OF EMBODIMENTS

Proteins, such as specified risk materials (SRM), canola protein, albumin, gelatin, blood meal protein and meat and bone protein are all examples of renewable proteins which can be modified as described herein, to produce a protein based flocculant material that can be useful in the coagulation and flocculation of water colloidal suspensions, for example MFT. SRM, blood meal protein and meat and bone protein are protein materials that can be derived from the waste stream in the animal rendering industry.

Proteinaceous materials can be recovered from SRM or similar materials from the rendering industry through known processes. In general amino acid analysis of the native protein materials shows that they are poorly soluble in water, and contain a high concentration of collagen (between 15 and 25% of the total protein content depending on sample). This is expected since collagen is the primary proteinaceous component of bones and cartilages, which make up the bulk of meat and bone meals.

Hydrolytic treatments can be used on the renewable protein materials in order to improve the solubility of the material in water. These treatments can include the use of heat, alkali, enzymes or a combination of the above. The general outcome of these treatments can be the reduction of the molecular size of the starting proteinaceous material accompanied by a varying degree of solubility which can facilitate subsequent recovery and utilization.

The hydrolytic protocols identified herein represent a substantial departure from the methods described in the prior art with regards to the temperature, pressure and residence time in the reactor vessel. One objective of the method when used in the modification of SRM is the destruction of prions, which have highly stable structures resistant to mild (low temperature and alkali concentrations) processing conditions, which are typically use when processing other waste proteins. Therefore, significantly more aggressive processing methods are used and described herein. In some embodiments, the methods, which can successfully destroy prions, can also fundamentally alter the nature of the native proteinaceous materials. The process can hydrolyze down to the amino acid or dipeptide level.

Molecular weight analysis can reveal that an alkaline hydrolysis protocol can cleave proteins more severely than thermal hydrolysis. Both methods can narrow the molecular weight distribution of the proteins and increased their solubility in water. An analysis of the non-protein-like residues post-hydrolysis can identify the presence of short-chain acids, amides, ammonia and other nitrogen compounds. These constitute a signature that alongside with scission of the peptide bond (the key element in polypeptide structures) other reactions occurred which fundamentally transformed the native proteins in materials that no longer maintained the key features of peptides.

After hydrolysis the modified/hydrolyzed protein material can be further modified by esterification, in order to ionize the terminal carboxylic acid groups, using, for example methanol, ethanol, propanol, a low molecular weight alcohol or other short chain alcohol. The process of esterification can take approximately 24 hours (for example when using methanol) or up to approximately a week (for example when using ethanol or propanol) and can be performed under acidic conditions, far example approximately 0.05 HCl. This reaction can be carried out and carefully controlled near room temperature without the aid of catalysts and can be driven to completion in reasonably short times (hours) depending on the starting concentration of the alcohols. Where desired this reaction can be quenched to attain only partial esterification.

An ester group can be electrically neutral in both acidic and alkaline pH ranges. The elimination of the carboxyl group on the modified protein material can reduce the amount of negative charges that can be present in alkaline pH ranges. As described in more detail below, this can allow the interaction of the modified protein material with the dispersed solids, which generally carry a negative charge.

Alternatively, or in addition, the modified protein material can undergo cationization, for example using 3-chloro-2-hydroxypropyl trimethyl ammonium chloride. Direct flocculation using polycation polymers can be used rather than inorganic coagulants because it can be safer and cleaner and produce less amount of sludge. Further, cationic peptides can contain positive centers regardless of the pH of the effluent stream and therefore can be more effective as a direct flocculating agent, without the need for a coagulant, in a wide pH range.

Further in the alternative, or in addition to any or all of the treatments described above, the modified protein material can be polymerized, for example by grafting peptides onto water soluble polymers which can extend networks in three dimensions and could trap charged particles. In some embodiments the peptides can be grafted onto straight chain polymers with various linker groups, for example poly(vinyl alcohol) or starch. Alternatively, the peptides can be grafted onto star-shaped organic molecules with various linker groups. In other embodiments, polyethylene glycols of varying molecular weights, acrylamide, isopropyl acrylamide and other derivatives of acrylamide can be used to polymerize the modified protein material. Using this process to increase the molecular weight of the modified protein material can result in larger and/or more stable flocs being formed. This can be useful when using SRM as the starting point of the modified protein material, as they are generally low molecular weight peptides.

The modified protein material can also be polymerized through self-polymerization. Coupling agents, which form linkages between two functional groups (—COOH and —NH₂) can be used to link together separate modified proteins. Further, polar functionally can be added to these self-polymerized modified protein materials in order to improve its ability to dissolve in water.

The modified protein materials described above can be used as a flocculant and/or coagulant in the treatment of waste water colloidal suspensions, for example mature fine tailings.

Flocculation and coagulation of solid dispersions, discussed herein, can be controlled by (among other factors) the distribution of electrical charges on the flocculant/coagulant. In turn, these can contribute to the solubility of the same compound in aqueous environment. Typically, within the usual pH range of waste water, most suspended solids carry a negative surface charge and repel each other. As a result, the suspended solids can be colloidally stable and resistant to aggregation. Therefore, materials with a positive charge can be used to destabilize the colloidal particles by charge neutralization thereby allowing coagulation of the suspended solids. This can also lead to the formation of slow settling microflocs.

When a flocculating agent is added the coagulated particles, or slow settling microflocs, themselves can aggregate, mainly by the formation of particle-polymer-particle bridges, resulting in larger and denser flocs, which can settle easily.

In some embodiments the protein material, modified using any or all of the herein processes and treatments, can act as both a coagulant and a flocculant and can be termed a modified protein based flocculant material, which can be used for the coagulation and flocculation of waste water colloidal suspensions, for example mature fine tailings. In other embodiments the modified protein based flocculant material can act mainly as a flocculant. In such instances the treatment of waste water colloidal suspensions can also involve the use of a coagulant, for example CaCl₂. The composition of the modified protein based flocculant material and coagulant can be used in the coagulation and flocculation of mature fine tailings and other waste water colloidal suspensions.

The process of coagulation and flocculation is well known in the art. In general, it involves providing a flocculant and mixing it with a colloidal suspensions. Flocs, which can develop in the mixture as a result of the action of the flocculant, can be allowed to settle. The flocs can also be removed using filtration. In some embodiments the pH of the modified protein material, acting as a flocculant, can be adjusted to approximately 4.

The bio-flocculants produced by the methods described herein can be biodegradable and have not been shown to produce any negative environmental impact. Once hydrolyzed, the resulting peptides are safe to release into the environment, as per the Canadian Food and Inspection Agency regulations, and have been shown in animal models to not have any infective ability.

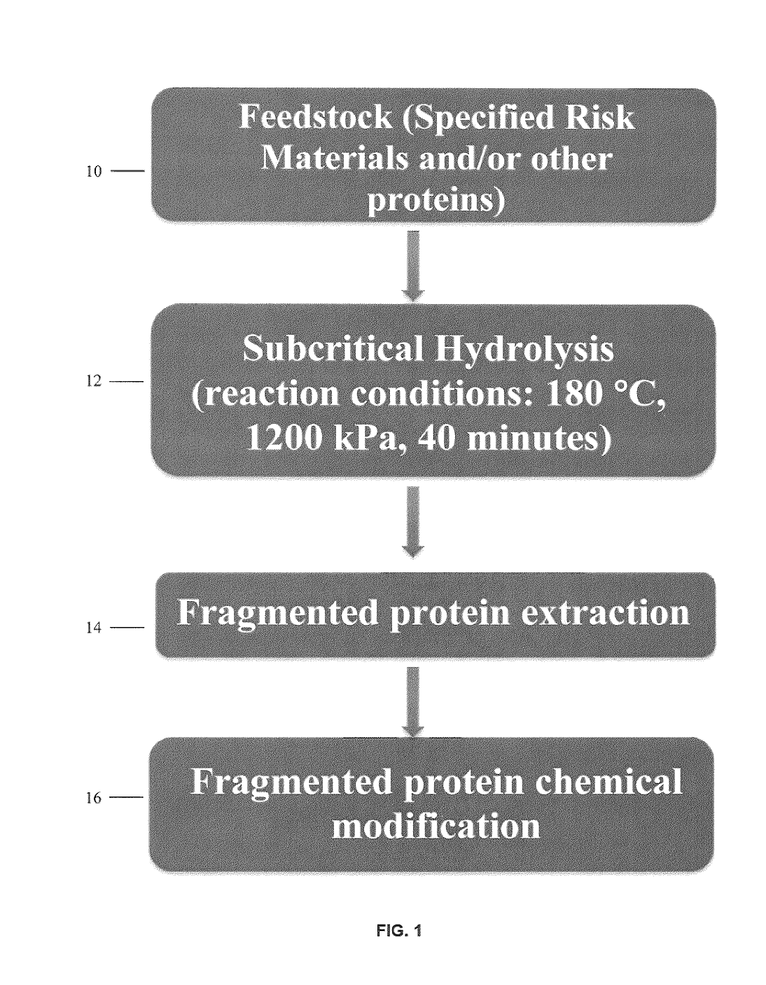

The process schematics presented in FIG. 1 summarizes an embodiment of a method to produce the materials and compositions described here. Renewable protein material can be obtained (10) for one of a variety of sources identified above. Next, hydrolysis can be performed (12) on the protein material to increase water solubility and reduce the molecular size of the starting material. In the case of SRM, the hydrolysis process can destroy the prion structure so that they are considered safe. These fragmented proteins can be extracted (14) and then they can undergo further protein chemical modification (16).

Without any limitation to the foregoing, the present methods, materials and compositions are further described by way of the following examples.

### Example 1

### Materials and Methods

Hydrolysis of Specified Risk Material (SRM): In some embodiments, SRM can be hydrolysed by thermal hydrolysis conducted at a minimum temperature of approximately 180° C. and pressure of at least approximately 1200 kPa for a period of approximately 40 minutes per cycle in an enclosed pressure vessel that is suitable for the purpose required. Such hydrolysis can destroy prion agents and can also show significant improvement for the solubility of the rendered SRM (T H Mekonnen, P G Mussone, N Stashko, P Y Choi, C Bressler, Recovery and characterization of proteinacious material recovered from thermal and alkaline hydrolyzed specified risk materials, Process Biochemistry 48 (5) (2013) 885-892, incorporated herein by reference) and other rendering products such as blood meal and meat and bone meal. Isolation of proteins from the hydrolyzed SRM, and isoelectric precipitation point determination can be conducted as per Mekonnen et al., (2013), This protocol can be used to prepare the protein hydrolysates used in the coagulation/flocculation.

Protein solution preparation: Hydrolyzed SRM protein isolates and all other unprocessed protein materials can be solubilized in milli-Q water at 100, 250, 400 and 500 ppm concentrations and 0.2 mM of CaCl₂ can be added. Divalent cations, such as CaCl₂ can help formation of bridges between the negatively charged clay particles to residual negatively charged functional groups of the protein based flocculants. The isoelectric precipitation point of gelatin and hydrolyzed SRM proteins were 5.5 and 4.5, respectively. Thus, the pHs of the formulated protein based flocculants can be adjusted to 4, so that they maintain a net positive charge.

Use of protein including SRM for flocculating model clay: Fine clay (Kaolin) solution can be used as a model for mature fine tailings flocculation. 10 mL of clay solution, consisting of 10% by weight of the model clay, was transferred to a 15 mL centrifuge. Performed in triplicate, 100, 250, 400 or 500 ppm of anionic polyacrylamide (PAM), gelatin or hydrolyzed SRM was then added and mixed at 300 rpm in a shaker for 10 min. The tubes were then placed in a tube rack, and left undisturbed while the contents settled at room temperature. At one, four, eight and twenty four hour, 0.5 mL of aliquots were taken about 2 cm below the surface of the solution, diluted and UV absorbance was measured at 600 nm. Absorption was then converted to clay suspension (mg/mL) with the aid of calibration curves prepared with a range of clay suspensions.

Flocculation of model clay: 500 ppm of commercial anionic polyacrylamide, gelatine or animal protein derived from hydrolyzed specified risk material (SRM) was prepared in distilled water. 100, 250 and 400 ppm of each of the prepared protein solutions were mixed in Erlenmeyer flasks with the oil sand tailings, that has a total solid content of about 27% by weight. The mixing took place in a shaker at 300 rpm for 10 min. Post mixing the contents were transferred to 100 mL volumetric measuring cylinders and the water ejected were observed every half hour for the first 2 hours, then every hour for the next 5 hours and after 24 hour.

Flocculation of mature fine tailings (MFT): 100, 250 or 400 ppm of PAM, gelatin or hydrolyzed SRM protein were added to a total volume of 100 mL MFT that contains 10% by weight of solids and mixed at 300 rpm for 10 min in a shaker. Each of the prepared MFT with the added flocculants were then transferred to 100 mL volumetric cylinder tubes and left undisturbed while the contents settled at room temperature. Water ejection was observed and recorded at 3 hr, 6 hr, 24 hr and 48 hr time points.

Turbidity and particle size distribution of aliquots: To quantitate the settling of particles in the ejected water, turbidity was measured using Nephelometer turbidometry (HACH 2100AN turbidometer). The size distribution of the particles left in the ejected water and the residue was also measured after the 48 h time point using Beckman Coulter™ Counter.

### Example 2

### Results

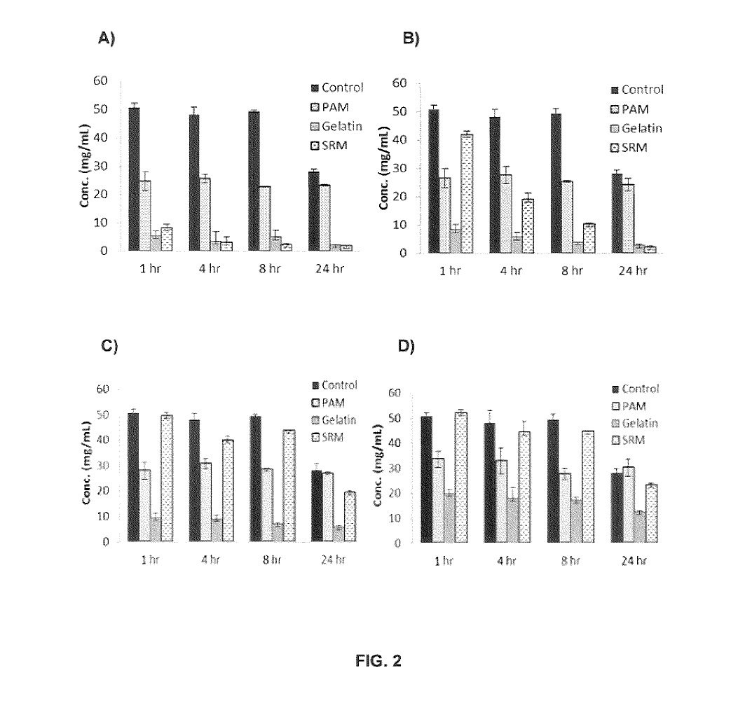

Flocculation of model clay: FIG. 2 shows the flocculation activity of anionic PAM, gelatin and hydrolyzed SRM at different concentrations as a function of time. It was observed that flocculation at 500 ppm of each flocculant was more efficient than the rest of the studied concentrations. Overall, gelatin protein exhibited higher flocculation activity than the hydrolyzed SRM. This can be attributed to the larger molecular size (>100 kDa) of gelatin than the hydrolyzed SRM (˜13 kDa). Moreover, the isoelectric precipitation point of gelatin (5.5) was higher than hydrolyzed SRM (4.5). This means gelatin protein might be more positively charged than hydrolyzed protein at pH 4, which again contribute to more flocculation of the negatively charged clay suspensions. Flocculation activity of proteins is usually observed at pH values lower than the protein isoelectric point, indicating a need for the protein to have a net positive charge (G J Piazza, R A Garcia, Proteins and peptides as renewable flocculant, Bioresource Technology 101 (2010), 5759-5766, incorporated herein by reference) to attach to the negatively charged clay. It is clearly observed that the gelatin at pH 4 performs better than PAM at all concentration ranges

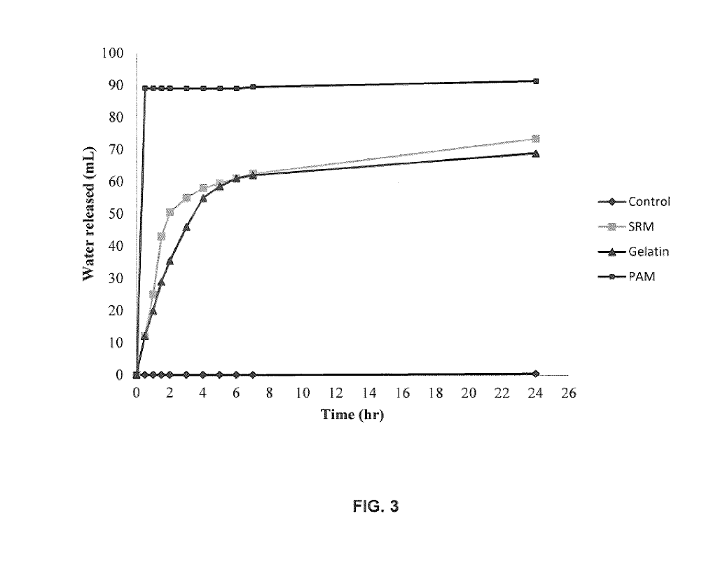

Tailing water release: The release of water by the mature fine tailing is depicted in FIG. 3. The PAM flocculation released about 89 mL of water in the first 30 min and stays constant for the remaining 6 hrs. After 24 hr sedimentation, the total water volume released by PAM was 91.5 mL. The SRM protein and gelatine released water in a linear fashion for the first 2 hrs and 3 hrs, respectively and the rate slowed down then after.

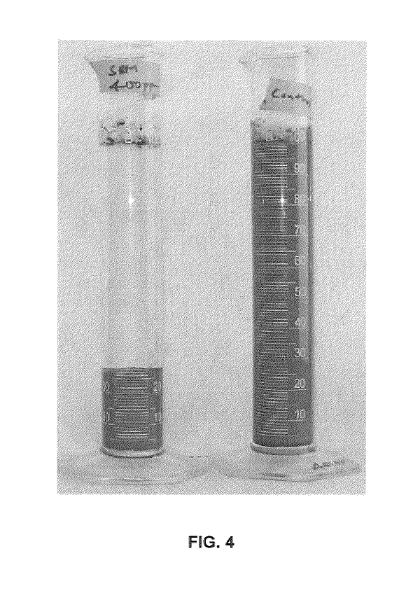

The PAM released higher volumes of water than both gelatin and SRM at the end of the 24 hours. However, the turbidity of the released water as measured by Nephelometer turbidometry, shown below in Table 1, was by far clearer for the SRM and gelatin than PAM. The control, with no addition of flocculant, did not release any water within the recorded 24 hours. These results can also been seen in FIG. 4.

TABLE 1
Turbidity of water released by the flocculants

| Flocculant | Turbidity (NTU) |
|---|---|
| SRM | 10.8 ± 0.2* |
| Gelatine | 858 ± 9* |
| PAM | 6825 ± 11* |

*Value ± Standard Deviation

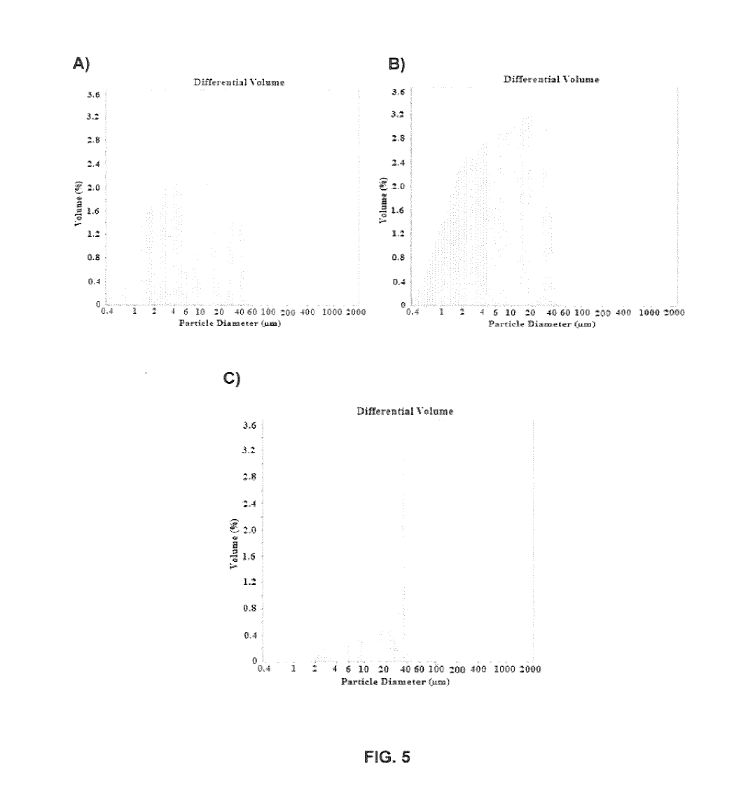

FIG. 5 shows the particle size distribution of the A) original oil sand tailing and after flocculation with hydrolyzed SRM B) the sedimented residue and C) the water released. The results show that the tailing and the sediment after flocculation contains particles over a wide range (0.4 to 60 □m). Hydrolyzed SRM flocculation settles most of the smaller particles, especially particles between 0.4 and 4 □m, which appear to be almost completely sedimented (FIG. 5C). Small volume fractions of the bigger particles were observed in the ejected water.

### Example 3

As an example of some embodiments, a detailed protocol for esterification can be summarized as follows.

An aliquot of protein fragments (typically 20 g of dry material) are dispersed in 500 ml of methanol solution containing 0.05 M HCl in 1 L Erlenmeyer flasks. The reactant solution is stirred at least 150 rpm for a period of time varying between 12 and 24 h at room temperature. The reaction time is a process variable that is directly correlated to the degree of esterification. Longer reaction times favour a higher degree of esterification. At the end of the reaction time, the methylated protein fragments are collected by centrifugation at 6000 rpm for 20 min and washed twice with HCl solution (0.05 M). The precipitated proteins are freeze dried. The methylated proteins are easily re-dissolved in water under ultrasonication (20 kHz, 30 W) for 20 min before use. The esterification of SRM and other hydrolyzed protein fragments can be accomplished using other short chain alcohols such as ethanol and butanol using the same protocol. However, as the carbon chain length is increased, the reaction times necessary to obtain industrially relevant yields are increased up to 48 hours in the case of butanol. Esterification with longer chain alcohols can result in substantial reduction of solubility of the protein fragments and is therefore not ideal.

### Example 4

To evaluate the effectiveness of peptides recovered from SRM as a flocculant and establish a benchmark for future studies, the effects of pH (5.5-8.5), concentration of peptides recovered from hydrolyzed SRM (1000-9000 mg/L) and the concentration of a coagulant (500-2000 mg/L calcium chloride, CaCl₂) on the flocculation of a kaolin clay model system using response surface methodology were investigated.

The peptides were esterified with methanol (24 hrs), ethanol (1 week) and propanol (1 week) under acidic conditions (0.05 M HCl). The modified peptides were recovered by centrifugation and air dried.

The peptides were cationized with 3-chloro-2-hydroxypropyl trimethyl ammonium chloride (1:1 by weight) at 50° C. for 18 hours.

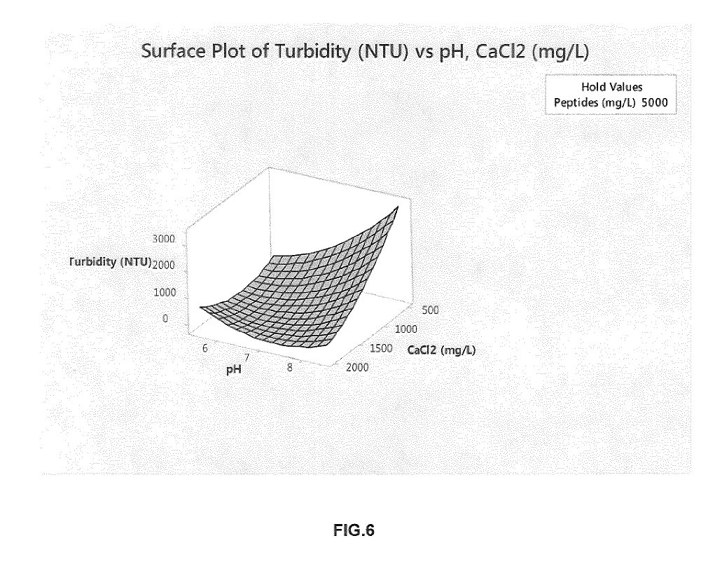

The flocculating ability of unmodified peptides recovered from SRM was studied using a kaolin clay model system. FIG. 6, which considers the effect of pH and CaCl₂ concentration on the turbidity of kaolin clay suspensions, shows that high concentrations of calcium chloride and alkaline pH result in low turbidity. It is typically found that use of a coagulant, such as iron or calcium salt, along with a flocculant produces less sludge. In this case CaCl₂ was used as a coagulant. The Ca(II) ions destabilize the colloidal particles by charge neutralization, and the subsequent aggregation of particles forms slow settling microflocs.

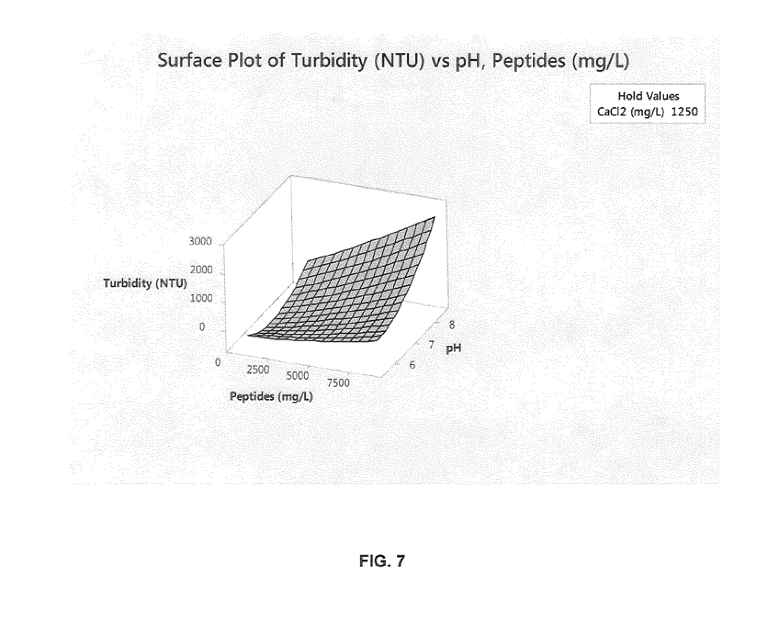

FIG. 7 considers the effect of peptide concentration and pH on the turbidity of the kaolin clay model system. It can be seen in FIG. 7 that lower concentrations (<2500 mg/L) of the peptides seen to have performed better, lowering the turbidity below 1000 NTU. Further, the peptides were found to be more effective in acidic pH, likely due to an increase in the positive centres as a result of the conversion of the terminal amine groups to ammonium ions in acidic pH, and these positive centres are effective in the removal of suspended solid particles through charge neutralization followed by floc formation.

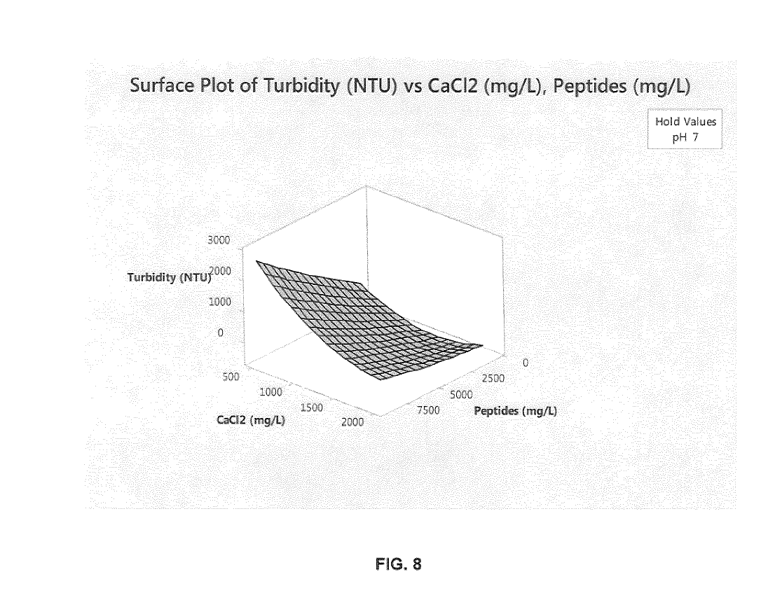

When the effect of the CaCl₂ concentration and peptide concentration were considered (FIG. 8), the turbidity is seen to decrease as the concentration of CaCl₂ increases, but the turbidity increases as the concentration of the peptide increases. This suggests that when the peptide is above a certain concentration (>2500 mg/L), it is contributing to the turbidity of the model system.

### Example 5

In order to test the biodegradability of hydrolyzed SRM, ˜2 g of epoxy cured TH SRM plastics were buried in natural soil and autoclaved soil. Control plastic samples (APS cured epoxy resin plastics) were also buried

The natural soil buried sample was kept in the department greenhouse and watered every day until the weight loss was measured. The autoclaved soil buried samples were set up containing moisture and sealed to avoid mass transfer from the outside environment.

After one month and three months the samples were dug out from the soil, debris was removed from the surface, and dried before measuring the weight. The weight loss of the plastics in both the natural and autoclaved soil was then calculated.

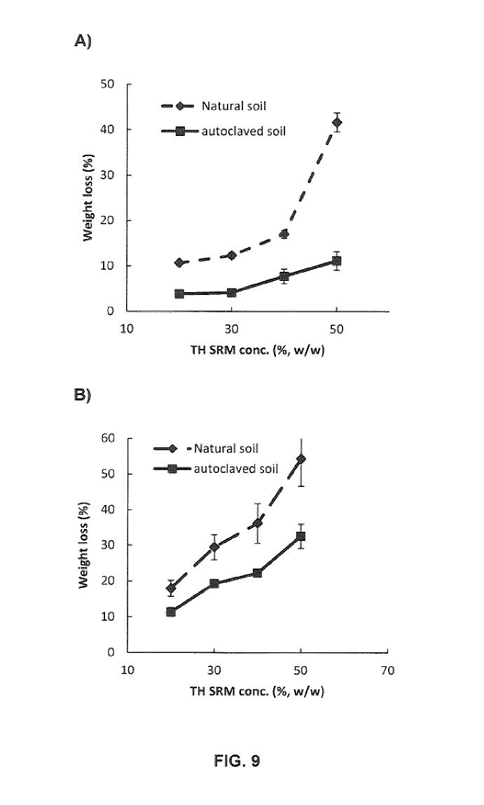

As seen in FIG. 9 the weight loss study exhibited that both biotic and abiotic factors contribute to the degradation of the protein based plastics. An incremental weight loss/degradation was observed as the plastics composition which contained more proteinaceous material. The control samples (diamine cured epoxy plastics) exhibited negligible weight loss (below the detection limit of the balance).

The scope of the claims should not be limited by the embodiments as set forth in the examples herein, but should be given the broadest interpretation consistent with the description as a whole. While only examples for SRM and gelatin are provided, canola protein, albumin, blood meal protein and meat and bone meal protein are anticipated to have similar characteristics after being processed using the disclosed hydrolysis process because the molecular structure of these proteins would all be similar after processing. The nature of the hydrolysis treatment can result in the end products from each of these protein sources being similar amino acid/dipeptide mixtures.

Although a few embodiments have been shown and described, it will be appreciated by those skilled in the art that various changes and modifications can be made to the embodiments described herein. The terms and expressions used in the above description have been used herein as terms of description and not of limitation, and there is no intention in the use of such terms and expressions of excluding equivalents of the features shown and described or portions thereof, it being recognized that the invention is defined and limited only by the claims that follow.

The teachings provided herein can be applied to other methods, materials and compositions, not necessarily the methods, materials and compositions described herein. The elements and acts of the various embodiments described above can be combined to provide further embodiments.

These and other changes can be made to the invention in fight of the above description. While the above description details certain embodiments of the invention and describes certain embodiments, no matter how detailed the above appears in text, the invention can be practiced in many ways. Details of the method can vary considerably in their implementation details, while still being encompassed by the invention disclosed herein.

Particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific embodiments disclosed in the specification. Accordingly, the actual scope of the invention encompasses not only the disclosed embodiments, but also all equivalent ways of practicing or implementing the invention.

The above description of the embodiments of the invention is not intended to be exhaustive or to limit the invention to the precise form disclosed above or to the particular field of usage mentioned in this disclosure. While specific embodiments of, and examples for, the invention are described above for illustrative purposes, various equivalent modifications are possible within the scope of the invention, as those skilled in the relevant art will recognize. The elements and acts of the various embodiments described above can be combined to provide further embodiments.

While certain aspects of the invention are presented below in certain claim forms, the inventor contemplates the various aspects of the invention in any number of claim forms. Accordingly, the inventor reserves the right to add additional claims after filing the application to pursue such additional claim forms for other aspects of the invention.

1. A modified protein material comprising:
hydrolyzed protein material selected from the group consisting of specified risk material (SRM), canola protein, albumin, gelatin, blood meal protein, and meat and bone meal protein, further comprising a controlled distribution of electrical charges;
wherein the hydrolyzed protein material is water soluble; and
wherein the hydrolyzed protein material acts as a flocculant of waste water colloidal suspensions.

2. The modified protein material of claim 1, wherein the hydrolyzed protein material has also undergone esterification using an alcohol.

3. The modified protein material of claim 2, wherein the alcohol is selected from the group consisting of methanol, ethanol, a low molecular weight alcohol, and other short chain alcohols.

4. The modified protein material of claim 1, wherein the hydrolyzed protein material has also undergone cationization.

5. The modified protein material of claim 1, wherein the hydrolyzed protein material has also undergone polymerization.

6. The modified protein material of claim 1, wherein the hydrolyzed protein material is biodegradable.

7. The modified protein of claim 1, wherein the hydrolyzed protein material acts as a coagulant.

8. The modified protein of claim 1, wherein the protein material is specified risk material.

9. The modified protein of claim 1, wherein the protein material is canola protein.

10. The modified protein of claim 1, wherein the protein material is albumin.

11. The modified protein of claim 1, wherein the protein material is blood meal protein.

12. The modified protein of claim 1, wherein the protein material is meat and bone meal protein.

13. (canceled)

14. A method to coagulate and flocculate waste water colloidal suspensions, the method comprising:
providing a flocculant comprising the modified protein material of claim 1;
mixing the flocculant with waste water colloidal suspensions to create a mixture; and
allowing mixture to settle;
wherein the waste water colloidal suspensions are coagulated and flocculated.

15. The method according to claim 14 further comprising filtration of the suspensions from the waste water.

16. The method according to claim 14 further comprising pre-treating the protein material to destroy prions.

17. The method according to claim 16 further comprising isolation of pre-treated protein material.

18. The method according to claim 14 further comprising adjusting the pH of the protein material to 4.

19. The method according to claim 14 further comprising mixing a coagulant with the flocculant and waste water colloidal suspensions to create a mixture.

20. A use of modified protein material of claim 1 for the coagulation and flocculation of mature fine tailings.

21. The use according to claim 20 wherein the protein material is a waste stream from the animal rendering industry.

22. (canceled)

23. (canceled)
